(19) United States
(12) Patent Application Publication
(10) Pub. No.: US 2025/0051704 A1
(43) Pub. Date: Feb. 13, 2025

(54) Multifunctional Petri Dish

(71) Applicants: Guo Feng Liu, An Shan (CN); Julie Zixuan Chen, Riverside, CA (US); Tian Li David Liu, Ithaca, NY (US); Xue Wu Liu, Bloomington, CA (US)

(72) Inventors: Guo Feng Liu, An Shan (CN); Julie Zixuan Chen, Riverside, CA (US); Tian Li David Liu, Ithaca, NY (US); Xue Wu Liu, Bloomington, CA (US)

(21) Appl. No.: 18/241,198

(22) Filed: Aug. 31, 2023

(30) Foreign Application Priority Data
Aug. 13, 2023 (CN) 202311016318.4

(51) Int. Cl. C12M 1/22; C12M 3/00

(52) U.S. Cl. CPC C12M 23/10; C12M 23/48; C12M 23/54

(57) ABSTRACT

The present invention discloses a multifunctional Petri dish with two sealing structures to ensure that the water in the medium will not evaporate during continuous culture, further affecting cultured living material growth and development. In addition to alcohol spray, the multifunctional Petri dish allows the application of acids, alkalis, alcohols, peroxides solution, or peroxides gas to immerse the multi-purpose Petri dish to remove bacteria or viruses. Various accessory devices can meet all requirements for a living culture of cells and tissues that the existing Petri dishes cannot provide. Using the dish cover operator and the dish container operator is essential to ensure that the researcher's hands will not directly touch the Petri dish in the sterile operating bench, thereby blocking all potential contamination of the medium and culture by foreign organisms. The labeling and labeling reading recorder with the multifunctional Petri dish provides more convenient and accurate electronic data establishment and reading for in vitro culture.

### CROSS-REFERENCES TO RELATED APPLICATIONS

This application claims the priority of P. R. China Patent Application, Application No. CN202311016318.4, filed on the date of Aug. 13, 2023.

### THE FIELD OF THE INVENTION

This invention is a multifunctional culture dish for tissues, embryos, cells, protoplasts, and microorganism cultures. It is the most widely used and essential experimental tool in life science, biology, education, bioengineering, and many other fields that cannot be listed individually.

### BACKGROUND AND RELEVANT ART

Petri dishes are the most important and irreplaceable experimental tools that are widely used in life science research, education, and bioengineering worldwide. However, from the birth of the Petri dish in 1887 to the present, water evaporation, air leakage, and contamination of the Petri dish have been long-standing problems in life research and bioengineering applications. Gerhard Brüsewitz (U.S. Pat. No. 4,675,298, table on page 2) disclosed that 50% of the water in agar evaporates within ten days in an unsealed Petri dish at 37° C. Although Parafilm, 3M Micropore, and other materials provide convenience and help for sealing Petri dishes, they still cannot effectively solve many problems, such as water evaporation and contamination of the culture medium. In biological culture experiments, manual marking of Petri dishes is a backward and cumbersome way. Although the widespread use of alcohol-resistant and other forms of marking pens is helpful for manual marking, it cannot be seamlessly linked with advanced electronic equipment, an obstacle for Petri dishes application. The prior art related to anti-evaporation, anti-contamination, medium replenishment, and other applications associated with Petri dishes can be found in U.S. Pat. No. 4,675,298 (Brusewitz and Gerhard), U.S. Pat. No. 4,160,700 (Boomus and Mary) and U.S. Pat. No. 3,660,243 (Young and Cecil). Chinese patents: CN 110283722 B (Cao Fanjing, Zhu Hao), CN202369595 (Qu Min), CN203212573U (Xiao Wei et al.), CN203794906U (Liu Yuan et al.), CN205741016U (Liu Jun and Xie Dan), CN204455118U (Zheng Guomin and Li Bo), CN206635333 (Gong Jian); Chinese patent application number: CN 20111047166.2 (Shi Shangli, et al.), CN 202011354847.1 (Qi Guoyou, et al.).

Currently, there are no sealable, anti-contamination Petri dishes with accessory devices and tools on the market that prevent water evaporation, especially no prelabeled multifunctional Petri dishes that are specially designed to remove oxygen or provide fresh air, nutrient supplementation, collect culture samples, and prelabeled. Therefore, there is an urgent need for anti-contamination, air-tight, water-tight, and more functions to meet the widespread and critical demand for multifunctional Petri dishes for life science research and bioengineering applications.

### BRIEF SUMMARY OF THE INVENTION

Implementations of the present invention overcome one or more problems by providing a multifunctional Petri dish to solve the problems of sealing the Petri dish, water evaporation, contamination, deoxygenation or provision of fresh air, nutritional supplementation, collection of culture samples, Petri dish labeling, etc. All these problems closely relate to life science research, biology, and engineering applications.

In one embodiment, implementations of the present invention overcome one or more technical problems in applying Petri dishes. The present invention adopts the following technical solution to provide a Petri dish with a first sealing structure and a second sealing structure. The functions of the two sealing devices are accomplished by the columnar structure or threads on the inside of the upright wall of the dish cover being screwed together with the threads on the outside of the vertical wall of the dish container. This screwing of the dish container and the dish cover causes the top of the upright wall of the dish container to press directly against the sealing ring on the dish cover, forming a first sealing structure that blocks water molecules from escaping from the agar medium or liquid culture medium. The screwing of the dish container and the dish cover causes the lower end of the upright wall of the dish cover to squeeze the second sealing ring placed on the circular stepped structure of the dish container, thus forming the second sealing structure. The formed second sealing structure has two essential functions: to provide further protection for the first sealing structure and to block various biological contaminants from further contaminating the culture in the Petri dish through the gap between the dish cover and the dish container. The Petri dish with the first and second sealing structures provides more options for sterilization of the Petri dish. The multifunctional Petri dish can be immersed in alcohol, acid, alkali, or highly oxidizing liquid to sterilize and disinfect the outer surface of the Petri dish and clean it in distilled water. This provides a technical guarantee and the possibility of achieving aseptic operation and sterile living culture.

In more particular embodiments, the dish cover and container have multiple accessory devices with specific functions, providing essential aids for the continuous living culture of tissues, cells, and microorganisms. Inject the culture into the dish through the first accessory device with specific functions to supplement the required nutrients, adjust the pH value of the culture solution, inoculate, replace aerobic or anaerobic gas. At the same time, the second accessory device can extract samples from the Petri dish to monitor the number of cells in the culture, the nutrition of the culture fluid, and the pH value, and release gas and pressure outside the dish. For example, filtered oxygen-free gases, such as nitrogen and carbon dioxide, are injected into the Petri dish through the first additional device. At the same time, the oxygen-containing gas in the Petri dish is released through the second accessory device to cultivate anaerobic bacteria, such as botulinum, providing an exceptional anaerobic culture environment. The multifunctional Petri dish with a third accessory device can use filter membranes or screen meshes to culture different biological species. The third accessory device is a channel for nutrient supplementation or replacement of the culture medium and sampling and monitoring the culture medium's pH, dissolved oxygen, and others. The various applications of the first accessory device, the second accessory device, and the third accessory device on the dish cover can significantly reduce the need to open the Petri dish, thereby effectively increasing the efficiency of the culture during the initial culture and the entire process of continuous culture. Effectively block contamination of cultures and media from biological sources of contamination. Since the first additional device, the second and third accessory devices can be applied directly after local disinfection, many intermediate links are significantly reduced, and sterile operating bench (box) use is reduced.

Furthermore, according to experimental requirements and culture scale, the multifunctional Petri dish can be converted from small to medium, large, or extra-large. The Petri dish's built-in atomizing nozzle, filter membrane, or screen mesh can provide a unique and required development and growth environment for animal and plant embryo culture, plant somatic embryo induction, plant regeneration, cell suspension culture, and microbial fermentation.

In more particular embodiments, the dish cover and container have multiple accessory devices with specific functions, providing essential aids for the continuous living culture of tissues, cells, and microorganisms. Inject the culture into the dish through the first accessory device with specific functions to supplement the required nutrients, adjust the pH value of the culture solution, inoculate, replace aerobic or anaerobic gas, etc.; At the same time, the second accessory device can extract samples from the Petri dish, perform real-time detection of the number of cells in the culture, the nutrition of the culture fluid, and the pH value, and release gas and pressure outside the dish. For example, filtered oxygen-free gases, such as nitrogen and carbon dioxide, are injected into the Petri dish through the first additional device. At the same time, the oxygen-containing gas in the Petri dish is released through the second accessory device to cultivate anaerobic bacteria, such as botulinum, providing an exceptional anaerobic culture environment. The third accessory device on the dish container can culture particular biological species using filter membranes or screens mash for nutrient supplementation or replacement of the culture medium, as well as sampling and monitoring the culture medium's pH, dissolved oxygen, etc. The various applications of the first accessory device, the second accessory device, and the third accessory device on the dish cover can significantly reduce the need to open the Petri dish, thereby effectively increasing the efficiency of the culture during the initial culture and the entire process of continuous culture. Effectively block contamination of cultures and media from biological sources of contamination. Many intermediate links are significantly reduced since the first, second, and third accessory devices can be applied directly after local disinfection. Furthermore, according to experimental requirements and culture scale, the multifunctional Petri dish can be converted from small to medium, large, or extra-large. The Petri dish's built-in atomizing nozzle, filter membrane, or screen mesh can provide a unique and required development and growth environment for animal and plant embryo culture, plant somatic embryo induction, plant regeneration, cell suspension culture, and microbial fermentation.

In more particular embodiments, the disclosures detail an efficient way to apply a barcode for labeling each Petri dish to solve the labeling problem that burdens biological culture. The barcode or QR code and Arabic numerals 0-9 constitute a unique label for each Petri dish, which solves the difficulties of setting and electronic reading of Petri dish labels. The label can track the quality of each Petri dish for manufacturers and can also be used to manage production and inventory quantities of Petri dishes. On the other hand, all the data on the Petri dish constitute a label that will never be repeated and can be directly used in biological research with the Petri dish as the basic unit. The label contains basic information covering the time a Petri dish was produced, including seconds, minutes, hours, day, month, year, and the Petri dish's serial number. People in the laboratory can use software to calibrate or remove certain parts of data on the barcode to make the required data shorter and more accessible. Barcodes can be scanned and recorded using mobile phones and laboratory-specific barcode readers and recorders, or the readable Arabic numerals can be read and recorded manually. All label data can be entered wirelessly, wired, or manually into a computer or data center.

Compared with the applications and technologies of existing Petri dishes, the present invention can comprehensively solve many problems, such as water evaporation and contamination in the application of Petri dishes, and meet all the needs of existing life science research and commercial biological living culture. The outstanding advantages of the present invention are described as the following:
1. The two sealing structures of the multifunctional culture dish fundamentally solve the problems of water evaporation and culture contamination in the culture dish, completely replacing the existing methods of sealing the culture dish using Parafilm, 3M CE Micropore, and other materials and techniques. Multifunctional Petri dishes can be immersed in alcohol, acid-base, and highly oxidizing liquids, such as potassium permanganate, sodium hypochlorite, etc., to sterilize and disinfect the external surface of the Petri dish.
2. The various accessory devices of multifunctional Petri dishes provide many functions for living biological culture that existing Petri dishes and culture technologies cannot achieve. All biological experiments can be performed under all conditions through effective local disinfection. For example, the replacement of oxygen or oxygen-free gas from a Petri dish, pH control of culture medium, monitoring of dissolved oxygen, nutritional supplement or replacement of culture medium, extraction of culture samples, used for real-time monitoring of cell growth during culture or biological fermentation. As well as single cells and bacteria transfer provide convenience and possibility.
3. The introduction and application of multifunctional Petri dish operators can stop both hands from touching the Petri dishes during the entire process in the sterile operating table (box), blocking the contamination from the culture operation. The specific structure of the multifunctional Petri dish and the particular structure design of the Petri dish operators match each other, providing an indispensable application guarantee in biological study. The dish cover and dish container operators can be exchanged with each other, which facilitates the operator's left- and right-handed operation habits and the adjustment of the operating angle and observation angle of the Petri dish in the sterile operating bench (box).
4. The label of each multifunctional culture dish has a dual role. It can provide the manufacturer of the multifunctional culture dish with full supervision of the quality and quantity of each one and also offer the end consumer a multifunctional culture dish with a global and unique label. This is the first example of a combination of barcode or QR code and Arabic numerals to provide manufacturers and end users with a globally universal and unique label and label sharing. Label can be read and recorded by any mobile phone with barcode or QR code scanning and reading capabilities or a laboratory-specific scanning and reading recorder. Arabic numerals can be used to read and records in case of sudden electronic equipment malfunction manually. All read data can be wirelessly wired or manually entered into a computer or data management center.

### BRIEF DESCRIPTION OF THE DRAWINGS

FIG. 1 is a composition diagram view of the multifunctional Petri dish;

FIGS. 2A to 2C are front view of the multifunctional Petri dish and plane views of the labels;

FIGS. 3A-3B are exploded view and partial enlarged view of the multifunctional Petri dish;

FIGS. 4A to 4J are various views of the dish cover operator and the detailed structural views of the dish cover operator;

FIGS. 5A to 5J are various views of the dish container operator and the detailed structural views of the dish container operator;

FIGS. 6A and 6B are exploded view and top front view of the first accessory and second accessory devices on the dish cover;

FIG. 7 is a front view of a multifunctional Petri dish with first accessory and second accessory devices;

FIGS. 8A-8C are top front views of the different specifications of multifunctional Petri dishes with the first, second, and third accessory devices.

### EXPLANATION OF NUMBERS IN THE DRAWINGS

100 multifunctional Petri dish, 101 dish cover, 102 dish container, 103 first sealing structure, 104 second sealing structure, 105 annular shallow groove, 106 label, 107 barcode reader and recorder, 201 barcode, 202 Arabic numbers, 203 PD (English abbreviation of Petri dish), 204 label paper, 301 upright wall of dish cover, 302 cylindrical structure, 303 threads, 304 first sealing ring, 305 second sealing ring, 306 thread, 307 upright wall of dish container, 308 circular stepped structure, 400 dish cover operator, 410 top part, 411 sheet structure, 412 end part structure, 413 positioner, 414 end structure, 415 narrow opening, 416 protective sleeve, 417 kink-like structure, 420 sandwiched part, 421 movable fixer, 422 sliding track groove, 423 spring, 424 screw nut, 425 first pull line, 426 pull lines tension adjuster, 427 stop bolt, 428, threaded tube, 429 pull line retraction control device, 430 knurled nut, 431 columnar protrusion, 432 fixing nut, 433 case, 434 first pull plate, 435 second pull plate, 436 pull rod, 437 upper hook, 438 upper part of the pull lines tension adjuster, 439 lower part of the pull lines tension adjuster, 440 lower hook, 441 penetrative holes, 442 cutaway portion, 450 bottom part, 451 manual lock switch button, 452 fixed frame, 453 movable frame, 454 pull line locking auxiliary structure, 455 pull line locking device, 456 spring, 457 pull line store tube, 458 second cable, 459 pressing line column, 460 second pull line fixing nut, 461 spring storage tube, 462 “L” shaped shallow groove, 463 lock lever, 464 lock spring, 465 spring pushing tube, 500 dish container operator, 510 top part, 511 sheet structure, 512 end part structure, 514 end structure, 515 narrow opening, 516 protective sleeve, 517 kink-like structure, 518 semicircular protrusion, 520 sandwiched part, 521 movable fixer, 522 sliding track groove, 523 spring, 524 screw nut, 525 first pull line, 526 pull lines tension adjuster, 527 stop bolt, 528, threaded tube, 529 pull line retraction control device, 530 knurled nut, 531 columnar protrusion, 532 fixing nut, 533 case, 534 first pull plate, 535 second pull plate, 536 pull rod, 537 upper hook, 538 upper part of the pull lines tension adjuster, 539 lower part of pull lines tension adjuster, 540 lower hook, 541 penetrative holes, 542 cutaway portion, 550 bottom part, 551 manual lock switch button, 552 fixed frame, 453 movable frame, 454 pull line locking auxiliary structure, 455 pull line locking device, 556 spring, 557 pull line store tube, 558 second cable, 559 pressing line column, 560 second pull line fixing nut, 561 spring storage tube, 562 “L” shaped shallow groove, 563 lock lever, 564 lock spring, 565 spring pushing tube, 601 left cone, 602 right cone, 603 first left square structure, 604 first right square structure, 605 left penetrative hole on the dish cover, 606 right penetrative hole on the dish cover, 607 second left square structure, 608 second right square structure, 609 first accessory device, 610 second accessory device, 611 circular mark, 700 multifunctional Petri dish with two accessory devices 800 multifunctional Petri dish with three accessory devices, 801 first accessory device, 802 second accessory device, 803 third accessory device, 804 circular mark.

To make the present invention's technical problems, solutions, and advantages to be solved by the present invention clearer, concise, and detailed, the outstanding features and benefits are now explained and introduced through the accompanying drawings. Although all Figs and descriptions focus on representative embodiments of the invention, this should not be considered or construed as limiting the scope thereof.

### DETAILED DESCRIPTION OF THE PREFERRED EMBODIMENTS

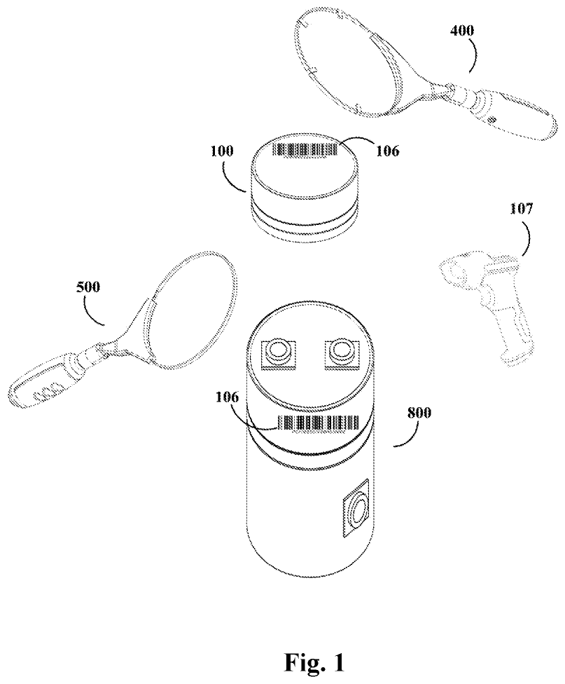

FIG. 1 is a front view of the composition of the multifunctional Petri dish. A multifunctional Petri dish includes a multifunctional Petri dish 100 or a multifunctional Petri dish with three accessory devices 800, a dish cover operator 400, a dish container operator 500, a label 106 for the multifunctional Petri dish, and a barcode reader and recorder 107 used to read the label on the multifunctional Petri dish.

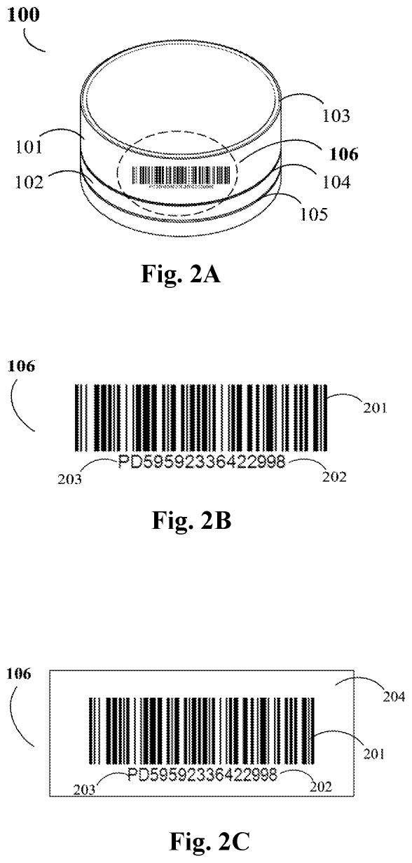

FIG. 2A is a front view of the multifunctional culture dish 100. A multifunctional culture dish 100 includes a dish cover 101, a dish container 102, a first sealing structure 103, a second sealing structure 104, an annular shallow groove 105, and a label 106 for the multifunctional culture dish label.

FIG. 2B is an enlarged view of the multifunctional culture dish label 106. The label 106 consists of barcode 201, PD 203, and Arabic numbers 202. Among them, PD 203 is the abbreviation of Petri dish. Arabic numerals include the production date and serial number. Among them, the month and day use 001 to 365, and the serial numbers of the Petri dishes start from 000 to 999, totaling 1,000. For example, the production date 59592336422, read from back to front, represents 23:59:59 on Dec. 30, 2022, and 998 represents the serial number of the Petri dish. The number of Petri dishes produced per second is 1,000, and the serial number starts from 000 Start and ends at 999. For example, label 106 can be interpreted as Petri dish No. 998 at 23:59:59 on Dec. 30, 2022. The next Petri dish is labeled PD59592336422999. The numerical portion of the label is constantly changing but at different frequencies, resulting in each Petri dish label being globally unique.

The PD 203 in the label can be replaced with the Petri dish model code or manufacturer's abbreviation. Petri dish manufacturers can use labels to monitor product quality and quantity. Barcodes can also be replaced with QR codes. Label 106 with barcodes, QR codes, and Arabic numerals 202 can be directly printed inside the upright wall or the dish cover 101. The barcode 201 or QR can also be printed on the label paper 204 (FIG. 2C) with self-adhesive adhesive to the inside of the upright wall 301 of the dish cover or the inside of the top edge. The Petri dish labels can be used directly as end-user labels. End users can use various app software to read and filter barcodes, such as reading and recording only the serial number of the Petri dish or the serial number plus seconds, minutes, months, and days. All mobile phones with scanning capabilities can read labels or use the lab-specific barcode reader and recorder 107 to read the label. All data can be input wirelessly or wired to a computer or data center. Under extreme conditions, the Arabic number part can be recorded manually and input into a computer or data center.

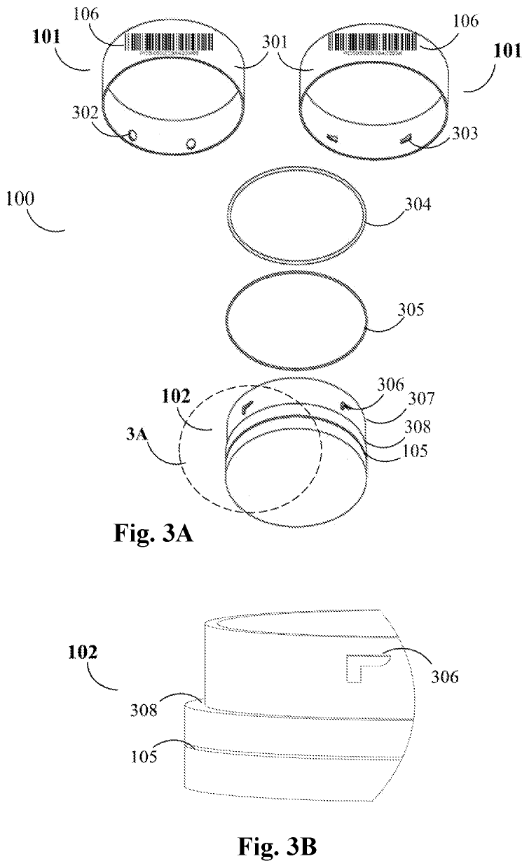

The exploded view (FIG. 3A) and the partially enlarged view (FIG. 3B) of the multifunctional culture dish 100 further demonstrate the structure of each part of the multifunctional culture dish 100 in detail. There are at least two cylindrical structures 302 or threads 303 on the inner wall of the upright wall 301 of the dish cover 101. The cylindrical structure 302 or threads 303 match the threads 306 on the dish container 102. The first sealing ring 304 and the second sealing ring 305 are the most essential structures among the first and second sealing structures. The outer upright wall 307 of dish container 102 has threads 306. The threads 306 is matched with the threads 303 or the cylindrical structure 302 on the dish cover so that the dish container 102 and the dish cover 101 can screw together. The surface of circular stepped structure 308 is used to place the second sealing ring. The annular shallow groove 105 below the circular stepped structure 308 is used to hold the semicircular protrusions 518 on the highly elastic protective sleeve 516 of the sheet structure 511 of the dish container operator 500. The annular shallow groove 105 has an obvious positioning function so that the semicircular protrusion 518 on the dish container operator 500 can firmly and accurately grasp dish container 102 without interfering with using the dish cover operator 400.

The detailed structure of the above diagrams of the dish container and the dish cover (FIG. 3A and FIG. 3B) shows that the first sealing structure 103 (FIG. 2) is composed of the edge of the top inner dish cover 101, the first sealing ring 304, and the top end of the upright wall 307 of the dish container. By twisting the threads 306 on the dish container with threads 303 or cylindrical structure 302 on the outer wall of the upright wall 307 to form a first sealing structure 103 that blocks water evaporation. Among them, the vertical line linked with threads 306 can prevent dish cover 101 and dish container 102 from being damaged due to over-screwing. The first sealing ring 304 is flat with thickness, can withstand high temperature and pressure, and can be reused. One side of the first sealing ring 304 has adhesiveness and is adhered and fixed to the top edge surface inside the dish cover 101.

The second sealing structure 104 comprises the surface of the circular stepped structure 308 (FIG. 3B) of the dish container 102, the second sealing ring 305, and the bottom end of the upright wall 301 of the dish cover 101. Threads 306 on the outer of the upright wall 307 of the dish container 102 can screw with the cylindrical structure 302 or threads 303 on the inner upright wall 301 of the dish cover 101 to form the second sealing structure 104. The formed second sealing structure 104 can completely block external biological contamination and further protect the first sealing structure. The second sealing ring 305 has the characteristics of resistance to acids, alkalis, alcohols, high oxides, resistance to high temperature, high pressure, and high elasticity, and can be used repeatedly. In addition to spraying alcohol on the surface for surface disinfection, the multifunctional Petri dish 100 can also be immersed in alcohol, acid, alkali, and high oxide liquids, such as potassium permanganate, sodium hypochlorite, etc., for external surface sterilization. Disinfect to remove specific acid- or alkali-resistant microorganisms and viruses that alcohol spraying cannot wholly kill. After the soaked Petri dishes are washed with distilled water, further aseptic operations required for in vivo biological experiments are performed.

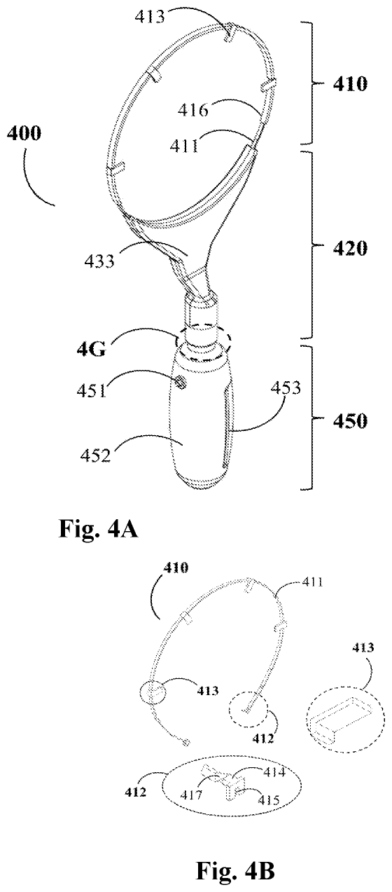

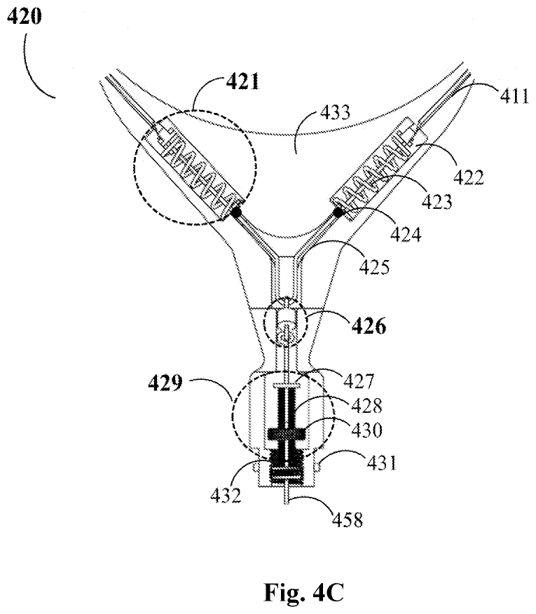

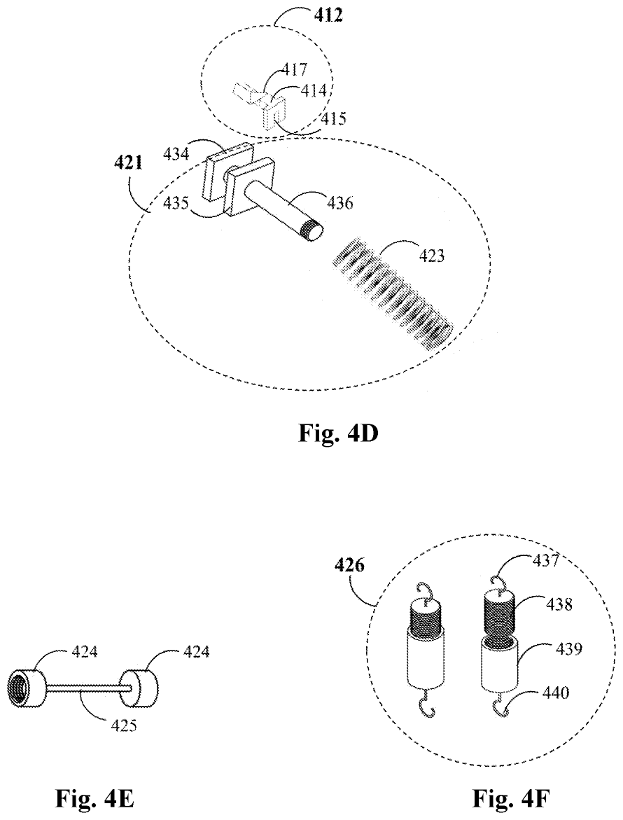

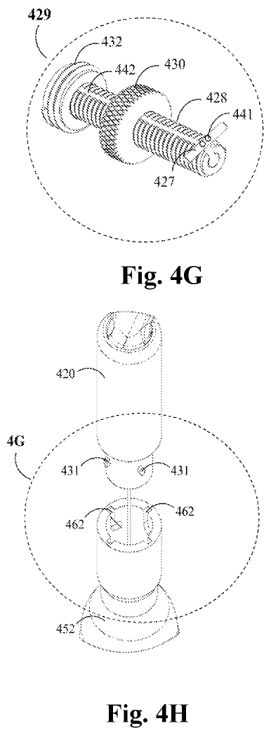

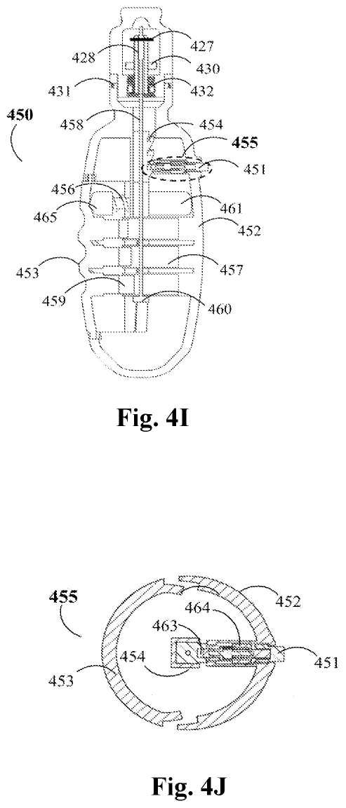

FIG. 4A is a front and three-dimensional diagram view of dish cover operator 400. The dish cover operator 400 comprises a top part 410, a sandwiched part 420, and a bottom part 450.

FIG. 4B is a front view of the top part 410 of the dish cover operator 400. The top part 410 comprises a rigid and elastic sheet structure 411 or other elastic alloy metal or material. The end part of structure 412 of sheet structure 411 is precisely shown in the partially enlarged view marked 412. There is a kink-like structure 417 at both ends of the sheet structure 411. The end part 414 of the kink-like structure 417 is bent ninety degrees with a narrow opening 415. The narrow opening 415 connects to the movable fixator 421 in the sandwiched part 420, which provides a simple and efficient way to integrate the top part 410 with the sandwiched part 420. The sheet structure 411 is completely wrapped by a highly elastic protective sleeve 416 to ensure that the sheet structure will not damage the dish cover. There are multiple positioners 413 on the sheet structure. The positioner 413 (see enlarged view inside the break circular line) can ensure that the sheet structure 411 on the top part of the dish cover operator 400 accurately grasps the dish cover 101. In particular, positioner 413 limits the sheet structure 411 of dish cover operator 400 for over-moving, controlling the dish cover operator 400 that never crosses the dish cover to interfere with dish container operator 500 for the dish container. The dish cover operator 400 can move the Petri dish to the operating area and place it on the top part 510 of the dish container operator 500 to open the Petri dish. The locking device 455 of the dish cover operator 400 can lock the dish cover 101 with the top part 410 of the dish cover operator 400 and place them outside the operating area of the sterile operating bench. Therefore, the hand that holds the dish cover operator 400 is free and can perform other procedures and tasks on the sterile operating table.

FIG. 4C is a plan view of the sandwiched part 420 of the dish cover operator 400. The sandwiched part 420 composes of a pair of movable fixers 421 used for connecting and fixing both end structures of the sheet structure 411; a sliding track groove 422 used to hold the movable fixers 421; a first pull line 425 with two screw nuts 424 used to link with the pull rod 436 in the movable fixers 421; a pull line tension adjuster 426 with two hooks used to connected with the first pull line 425 through the hook 437 and the second pull line 458 via hook 440. A pull line retraction length control device 429 on the second pull line 458 to control the maximum contraction range of the upper part 410 to grasp the Petri dish to ensure that the dish cover 101 will not be physically damaged due to the deformation energy of the spring 423. The pull line contraction length control device 429 consists of a stop bolt 427, a threaded tube 428, a knurled nut 430, and a fixing nut 432; the bottom end of the case 433 of the sandwiched part 420 has a columnar protrusion 431 used to twist with the “L” shaped shallow groove 462 on the top end of the lower part 450 of the dish cover operator. As a result, the sandwiched part 420 is integrated with the bottom part 450 into one.

FIG. 4D is an exploded view of the movable fixer 421. The movable fixer 421 comprises a first pull plate 434 with a threaded penetrative central hole, a second pull plate 435 with a central penetrative hole, a pull rod 436 with threads at both ends and a spring 423. The space between the first pull plate 434 and the second pull plate 435 is used to place the end structure 512 of the sheet structure 411. The narrow opening 415 spans the pull rod 436 so that the two ends of the sheet structure 411 can be tightly connected and bound to the movable retainer 421 under the action of the spring 423. This connection method results in the entire connection of the top part 410 with the sandwiched part 420. It also brings great convenience to replacing the top part 410 of the dish cover operator 400.

FIG. 4E is a left front view of the first pull line 425. The two ends of the first pull line 425 are screw nuts 424, which are used to screw with the pull rod 436.

FIG. 4F is the front view (left side) and exploded view (right side) of the pull line tension adjuster 426. The pull line tension adjuster 426 has two parts: the upper part 438 with hooks 437 and the lower part 439 with hook 440. The hook 437 is used to connect the first pull line 425. The hook 440 is used to connect the second pull line 458. The upper and lower parts of the pull line tension adjuster 426 are screwed together to adjust the tightness of the first pull line 425 and the second pull line 458. The pull line tension adjuster 426 ensures that the position of the stop bolt 427 is always at the edge of the threaded tube 428 (FIG. 4G) in the stationary state. The pull line tension adjuster 426 is also the first significant essential structure to disassemble the dish cover operator 400.

FIG. 4G is a left front view of the pull line contraction length control device 429. The pull line contraction length control device 429 is composed of a stop bolt 427 with two penetrative holes 441, a threaded tube 428, a knurled nut 430, and a fixed nut 432, wherein the cutaway portion 442 of the threaded tube 428 is the channel for the stop bolt 427 to enter the threaded tube 428. The two penetrative holes 441 on the stop bolt 427 allow the second pull wire 458 to pass. The pull line retraction length control device 429 can strictly control the length of the first and second pull lines tightening and the degree of tightening of the sheet structure 411 to ensure that the fragile Petri dish will not be damaged due to excessive tightening of the sheet structure. The distance between the stop bolt 427 and the knurled nut 430 is crucial. The basis for distance adjustment is to calculate the difference between the circumference of the relaxed sheet structure 411 and the known circumference of the Petri dish. Then, divide the difference by 2 to determine the distance between the stop bolt 427 and the knurled nut 430 distance.

FIG. 4H is a partially exploded view of the bottom end of the sandwiched part 420 and the top end of the bottom part 450 of the dish cover operator. The columnar protrusion 431 on the lower end of case 433 enters the “L” shaped shallow groove 462 on the top end of the fixed frame 452 and twists, resulting in the sandwiched part 420 and the bottom part 450 wholly connected. This connection method enables the top part 410 and the bottom part 450 of the dish cover operator 400 to be adjusted within 360 degrees through the sandwiched part 420. This facilitates aseptic operations by scientific researchers and operators on the sterile operating bench.

FIG. 4J is a cross-sectional view of the lower part of the sandwiched part 410 and the bottom part 450 of the dish cover operator 400. The lower part 450 of the dish cover operator 400 includes a fixed frame 452 with a spring storage tube 461, a spring 456, a pull line locking device 455, a pull line store tube 457, and a manual lock switch button 451. When the movable frame 453 is tightened, the movable frame 453 presses the spring, pushing tube 465 to result in the spring 456 moving toward the spring storage tube 461. At the same time, the presses the second pull line 458 through the line pressure columns 459 to enter the pull line store tube 457, which drives the pull line auxiliary locking structure 454 to move downward, thereby triggering the lock of the line locking device 455. When the stop bolt 427 meets the knurled nut 430, the lock rod 463 of the pull line locking device 455 is locked with the pull line auxiliary locking structure 454. Now, no matter how hard you hold the movable frame 453, the grip force will not act on the sheet structure 411 through the second pull line 458, protecting the culture dish from damage.

As shown in FIG. 4J, the pull wire locking device 455 is a device for the dish cover operator to lock the dish cover. When the second pull line is pressed into the take-up tube 457 by the line pressure column 459, the pull wire locking accessory structure 454 slides downward simultaneously, causing the stop bolt 427 to intersect with the knurled nut 430. At this time, the protrusion on the pull line locking accessory structure 454 triggers the locking rod 463 to eject from the lock device 455 and embed into the groove of the pull line locking accessory structure 454 so that the second pull line 458 is locked. This causes the upper part 410 of the dish cover operator 400 to be locked with the dish cover 101. At any time, the locked dish cover 101 can separate from the top part 410 of the dish cover operator by pressing the manual lock switch 451. The elastic force from the spring 464 results in the locking rod 463 retracted to the pull line locking device 455.

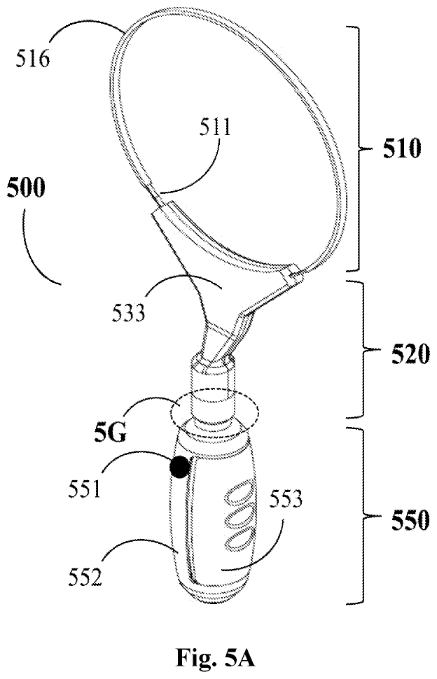

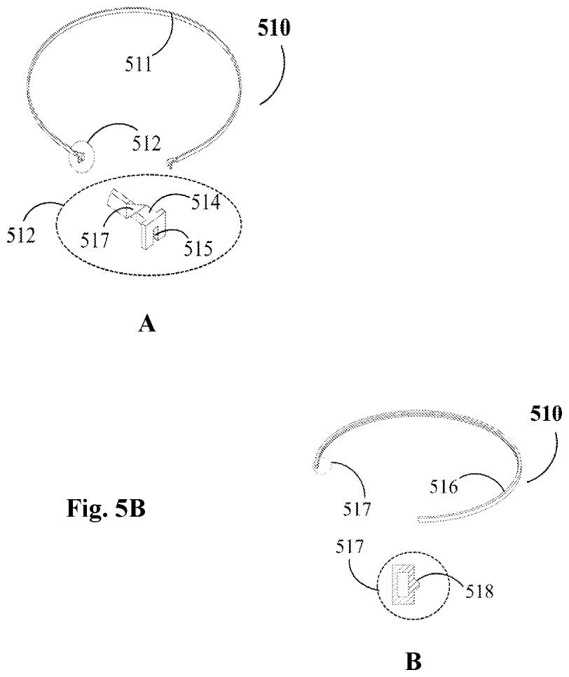

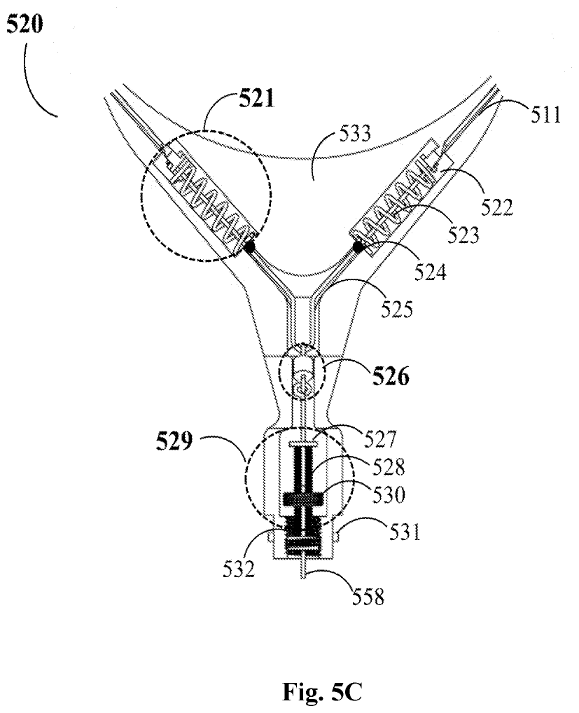

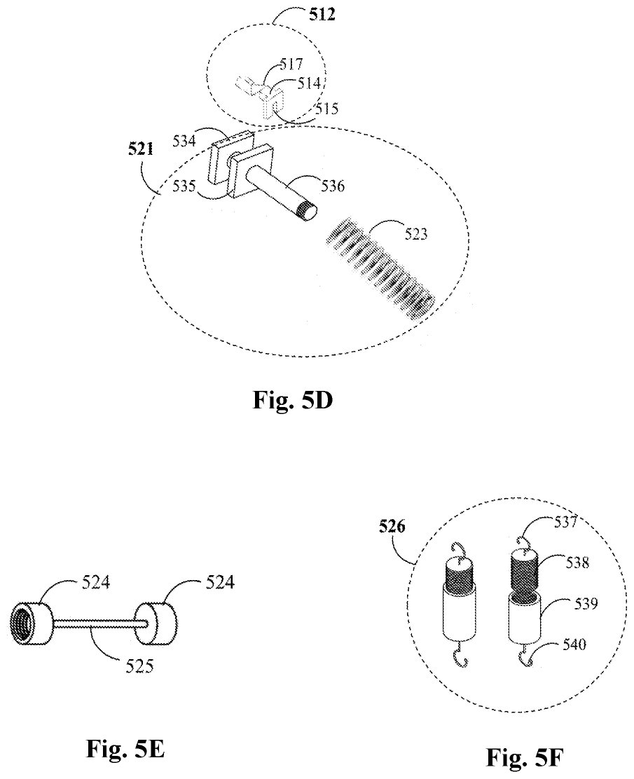

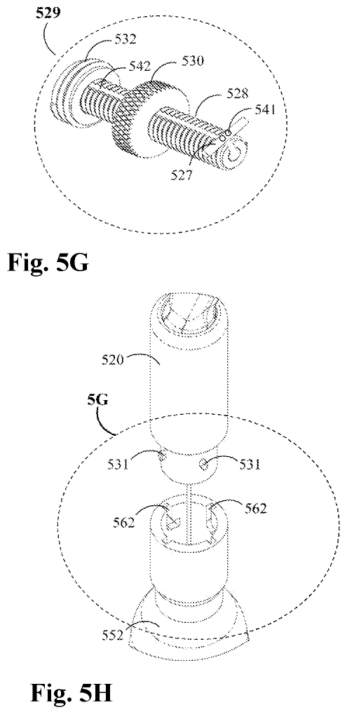

FIG. 5A is a front view of the dish container operator 500. The dish container operator comprises a top part 510, a sandwiched part 520, and a bottom part 550.

FIG. 5B is the top front views of the top part 510 of the dish container operator 500. As shown in A and B of the FIG. 5B, the top part 510 comprises a sheet structure 511 (A in FIG. 5B) and a highly elastic protective sleeve 516 (B in FIG. 5B). There is a kink-like structure 513 at each end of the sheet structure 511. The end part 514 of the kink-like structure is bent ninety degrees with a narrow opening 515. The narrow opening 515 at the end of the kink-like structure connects to the movable fixator 521 in the sandwiched part 520. There is a semicircular protrusion 518 on the inside of the highly elastic protective sleeve 516 with a buffering effect for engaging with the annular shallow groove 105 of the dish container 102. The semicircular protrusion 518 has an obvious positioning function so that the dish container operator 500 can properly grab the dish container without interfering with the dish cover operator 400.

The sandwiched part 520 (FIG. 5C to FIG. 5H) and the bottom part 550 (FIG. 5H and FIG. 5J) of the dish container operator 500 are the same structures as the sandwich part 420 and the bottom part 450 of the dish cover operator 400. Therefore, please refer to illustrations for FIGS. 4C, 4D, 4E, 4F, 4G, 4H, 4I, and 4J from paragraphs [0036] to [0042] to understand the structure and function of the sandwiched part 520 and the bottom part 550 of the dish container operator 500.

As shown in FIGS. 4 and 5, the dish cover operator 400 and the dish container operator 500 can be sterilized and reused using reusable high-pressure and high-temperature resistant sterilization bags. As shown in FIGS. 4A to 4I, any part of the dish cover operator 400 and the dish container operator 500 can be replaced without requiring special tools.

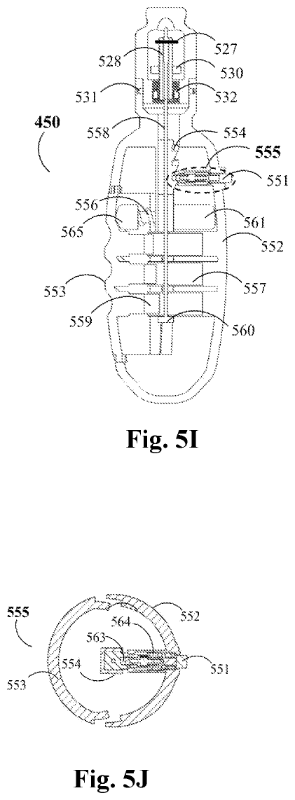

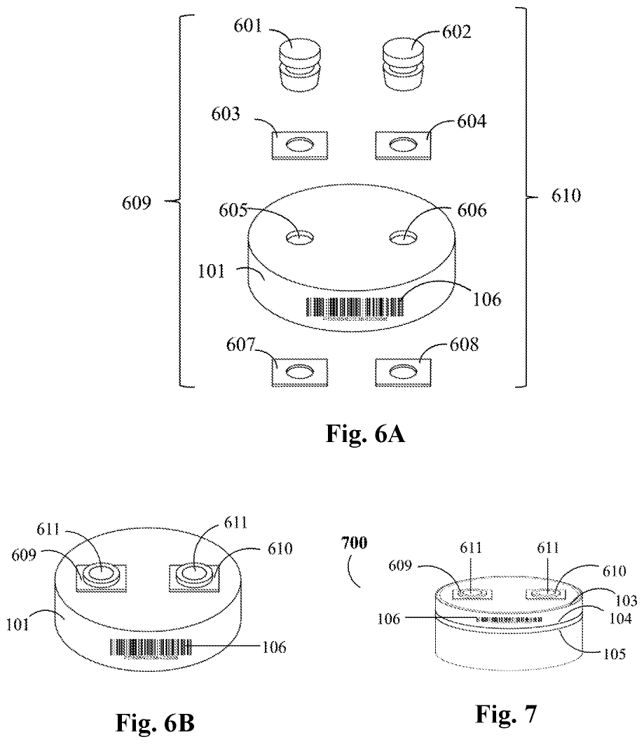

FIG. 6A is an exploded and front view of the dish cover 101 with first accessory device 609 and first accessory device 610. The first accessory device 609, located on the left side of the dish cover 101, consists of a cone 601 with a convex and concave structure, a rigid and thick square structure 603 with a penetrative hole, a penetrative hole 605 on the left side of the dish cover, and a rigid and thick square structure 607 with a penetrative hole. The square structures 603 and 607 are jointly glued with the penetrative hole 605 of the dish cover by non-toxic, high-temperature, and high-pressure resistant glue. The cone 601 is glued with square structures 603 and 607 to form a well-sealed first accessory device, 609. The second accessory device 610, located on the right side of the dish cover 101, consists of a cone 602 with a convex and concave structure, a rigid and thick square structure 604 with a penetrative hole, a penetrative hole 606 on the right side of the dish cover and a rigid and thick square structure 608 with a penetrative hole. The square structures 604 and 608 are jointly glued with the penetrative hole 606 of the dish cover 101 by non-toxic, high-temperature, and high-pressure resistant glue. The cone 602 is glued with square structures 604 and 608 to form a well-sealed second accessory device, 610.

The front view (FIG. 6B) is the dish cover 101 with the first accessory device 609, the second accessory device 610, and the label 106.

FIG. 7 is a front view of the multifunctional Petri dish 700 with the first sealing structure 103, the second sealing structure 104, the first accessory device 609, the second accessory device 610, and the label 106. Among them, the circular mark 611 on the first accessory device 609 indicates that the area inside the annular shape can be used to input enriched nutrient solution, filtered gas, bacteria, etc., into the multifunctional Petri dish through a sterile needle passing through the cone. The circular mark 611 on the second accessory device 610 allows the sterile needle to pass inside the annular area to extract cultured and culture media samples and release gas in the culture dish.

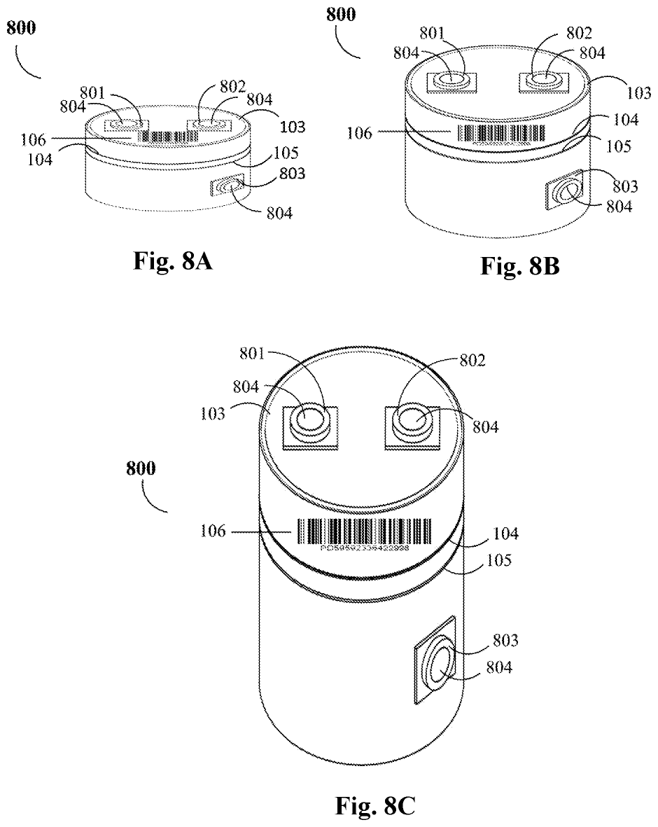

FIGS. 8A-8C are top front views of the multifunctional Petri dishes 800 of different specifications having first, second, and third accessory devices 801, 802, and 803. The circular mark 804 is a safe area in all accessory device applications, allowing needles and the like to enter the Petri dish within the ring mark. The multifunctional Petri dish 800 has three accessory device for particular cultures requiring a built-in filter membrane or screen mesh. Adding an accessory device 803 to the dish container can complete the injection of a culture medium or other liquids that can adjust the pH of the culture medium into the dish container without causing damage to the filter membrane or screen mesh. Particular attention should be paid to that when using the first accessory device, 801, on the dish cover, the second accessory device, 802, must be used at the same time, and filtered air is added into the dish container through a sterile needle and filter to avoid positive and negative pressure in the dish container and cause damage to the Petri dish.

The enlarged Petri dish 800 with the first accessory device 801, the second accessory device 802, and the third accessory device 803 allows a built-in spray head to provide the nutrient solution to the culture in the form of a spray. The enlarged multifunctional Petri dish 800 also allows other devices, such as temperature sensor, pH sensor, or other sensors related to living culture, to be placed inside the Petri dish. The multifunctional Petri dishes allow machines to automatically add culture medium to the Petri dish or/and replace the aerobic air from the Petri dish with filtered nitrogen or/and carbon dioxide gas through the accessory devices using sterilized needles and different filters through the accessory devices. The multifunctional Petri dishes allow machines to automatically transfer or add cells or strain through the accessory devices to the multifunctional Petri dish.

The above embodiments of a multifunction Petri dish, Petri dish operators, and labels are disclosed in all detail, with many figures from every angle showing the structures. Any person familiar with the technical field shall make equivalent substitutions or changes based on the present invention's technical solutions and improvement concepts within the technical scope disclosed in the present invention and shall be covered by the protection scope of the present invention.

What is claimed are:

1. a multifuntional Petri dish comprising:
a multifunctional Petri dish;
a pair of Petri dish operators used to hold, open, and close the multifunctional Petri dish when operating in a sterile operating bench or box;
a label used for the multifunctional Petri dish; and
a label reader and recorder;
wherein the multifunctional Petri dish is a disposable or reusable circular, rectangular or square container, including:
a dish cover,
at least one first sealing structure,
at least one second sealing structure, and
a dish container;
wherein the multifunctional Petri dish can be immersed in alcohol, acid, alkali, and highly oxidizing liquids for external surface sterilization and washed in distilled water;
wherein the pair of Petri dish operators used to hold, open, and close the multifunctional Petri dish, including:
at least one operator used for the dish cover;
wherein the dish cover operator comprising:
a top part,
a bottom part, and
a sandwiched part for linking the top part and the bottom part to form the dish cover operator;
at least one operator used for the dish container;
wherein the dish container operator container comprising:
a top part,
a bottom part, and
a sandwiched part for linking the top part and the bottom part to form the dish container operator; and
a reusable sterilization bag used to sterilize both of the operators under high temperature and high-pressure conditions.

2. The multifunctional Petri dish recited in claim 1, wherein the dish cover has at least one pair of cylindrical structures or one couple of threads on the inner upright wall.

3. The multifunctional Petri dish recited in claim 1, wherein the dish container has at least one pair threades on the outside of the upright wall, which screw with the cylindrical structures or threads on the inner upright wall of the dish cover;
wherein the lower part of the upright outer wall of the dish container has a circular stepped structure;
wherein the circular step structure has an annular shallow groove.

4. The multifunctional Petri dish recited in claims 1, 2, and 3, wherein the first sealing structure comprising:
a top inner edge of the dish cover,
at least one first sealing ring, and
a top end of the upright wall of the dish container;
wherein the threads on the upright wall of the dish container screw with the cylindrical structures or the threads on the inside of the vertical wall of the dish cover together to form the first sealing structure that blocks water evaporation;
wherein the top side of the first sealing ring connected to the top edge of the dish cover is sticky pasted on the edge surface of the inner dish cover.

5. The multifunctional Petri dish recited in claims 1, 3, and 4, wherein the second sealing structure having:
a top surface of the circular stepped structure on the outer wall of the dish container,
at least one second sealing ring fixed onto the top surface of the circular step structure, and
a bottom end of the upright wall of the dish cover;
wherein the threads on the upright wall of the dish container screw with the cylindrical structures or the threads on the inside of the upright wall of the dish cover together to form the second sealing structure that blocks water evaporation;
wherein the second sealing structure blocks external biological contaminants from contaminating the culture medium or cultured materials and further protects the first sealing structure.

6. The multifunctional Petri dish recited in claim 1, wherein the top part of the dish cover operator comprising:
a narrow and elastic sheet with kink-like structures at both ends;
a ninety-degree bent with a narrow opening on both ends of the kink-like structures;
at least two positioners on the top of the sheet; and
a protective sleeve to cover the sheet.

7. The multifunctional Petri dish recited in claim 1, wherein the bottom part of the dish cover operator having:
a handle with a fixed frame part; and movable frame part;
wherein the fixed frame part having:
at least one “L” shaped shallow groove on the top end;
at least one spring storage tube;
a plurality of the second pull line storing tubes fixed on the fixed frame; and
a locking device;
wherein the locking device having:
a locking rod;
a locking spring;
a locking auxiliary structure; and
a manual locking switch
wherein the movable frame having:
at least one spring pushing tube has a spring;
a plurality of the movable line pressing columns;
wherein the plurality of the second pull line storing tubes and the plurality of the movable line pressing columns constitutes a pulling line releasing and retracting device;

8. The multifunctional Petri dish recited in claim 1, wherein the sandwiched part of the dish cover operator comprising:
a pair of movable fixers;
a pair of sliding track grooves for the movable fixers;
a first pull line with two screw nuts at both ends;
a second pulling line;
a pulling lines tension adjuster with two hooks;
a pull line retraction length control device;
at least one columnar protrusion; and
a case;
wherein one of the movable fixers including:
a first pull plate with a threaded penetrative central hole;
a second pull plate with a penetrative central hole;
a pull rod with threads at both ends; and
a spring;
wherein the pulling line retracting control device comprising:
a quarter-opening threaded tube;
a fixing nut;
a knurled thumb nut; and
a stop bolt with two penetrative holes.

9. The multifunctional Petri dish recited in claim 1, wherein the top part of the dish container operator comprising:
a narrow and elastic sheet with kink-like structures at both ends;
a ninety-degree bent with a narrow opening on both ends of the kink-like structures;
at least two positioners on the top of the sheet; and
a protective sleeve to cover the sheet.

10. The multifunctional Petri dish recited in claim 1, wherein the bottom part of the dish container operator having:
a handle with a fixed frame part; and movable frame part;
wherein the fixed frame part having:
at least one “L” shaped shallow groove on the top end;
at least one spring storage tube;
a plurality of storing tubes for the second pull line; and
a locking device;
wherein the locking device having:
a locking rod;
a locking spring;
a locking auxiliary structure; and
a manual locking switch
wherein the movable frame having:
at least one spring-pushing tube with a spring;
a plurality of movable line-pressing columns;
wherein the plurality of the storing tubes for the second pull line and the plurality of the movable line pressing-columns constitutes a pull line releasing and retracting device.

11. The multifunctional Petri dish recited in claim 1, wherein the sandwiched part of the dish container operator comprising:
a pair of movable fixers;
a pair of sliding track grooves for the movable fixers;
a first pull line with two screw nuts at both ends;
a second pulling line;
a pulling lines tension adjuster with two hooks;
a pull line retraction length control device;
at least one columnar protrusion; and
a case;
wherein one of the pair of movable fixers having:
a first pull plate with a threaded penetrative central hole;
a second pull plate with a penetrative central hole;
a pull rod with threads at both ends; and
a spring;
wherein the pulling line retracting control device comprising:
a quarter-opening threaded tube;
a fixing nut;
a knurled thumb nut; and
a stop bolt with two penetrative holes.

12. The multifunctional Petri dish recited in claim 1, wherein the label used for a multifunctional Petri dish comprising:
a barcode or a QR code; and
Arabic numerals 0-9;
wherein the barcode or the QR code, and the Arabic numerals are printed on the inner upright wall or the inner top edge of the dish cover; or the barcode, or the QR code, and the Arabic numerals are printed on a sticky label paper and then posted on the inner upright wall or the inner top edge of the dish cover;
Wherein the label reading and recording device is a mobile phone or a laboratory-specific label scanning and recording device;
wherein the data obtained from the reading device(s) can be wirily or wirelessly transferred to a computer or a data center;
wherein the Arabic numbers can be recorded and input to a computer manually whenever the label reader is unavailable.

13. a multifunctional Petri dish comprising:
a multifunctional Petri dish with a plurality of volume specifications;
wherein the multifunctional Petri dish having:
at least one first sealing structure;
at least one second sealing structure;
a plurality of accessory devices with specific functions,
wherein at least two of the plurality of accessory devices are on the dish cover;
wherein at least one of the plurality of accessory devices is on the dish container;
a pair of Petri dish operators used to hold, open, and close the multifunctional Petri dish when operating in a sterile operating bench or box;
a label used for the multifunctional Petri dish; and
a label reader and recorder.

14. The multifunctional Petri dish recited in claim 13, wherein one of the two accessory devices on the dish cover is the first accessory device;
wherein the first accessory devices with specific functions having:
at least one cone structure with a convex-concave,
a circular mark on the top cone structure,
at least one first rigid and thick square structures with a penetrative holes,
at least one second rigid and thick square structures with a penetrative holes, and
at least one penetrative hole at one side of the dish cover;
Wherein the first accessory device with specific functions is used to inject nutrients required for culture, single cells or strains, pH adjustment liquid, and filtered air or nitrogen or/and carbon dioxide gas into the Petri dish through a medical sterile needle.

15. The multifunctional Petri dish recited in claim 13, wherein one of the two accessory devices on the dish cover is the second accessory device;
wherein the second accessory device with specific functions having:
at least one cone structure with a convex-concave,
a circular mark on the top cone structure,
at least one first rigid and thick square structures with a penetrative holes,
at least one second rigid and thick square structures with a penetrative holes, and
at least one penetrative hole at one side of the dish cover;
Wherein the second accessory device with specific functions is used to take samples from the multifunctional Petri dish used to monitor the cultured cell growth, development, pH, and dissolved oxygen in the culture medium, and also used to release air and pressure from the multifunctional Petri dish.

16. The multifunctional Petri dish recited in claim 13, wherein the one accessory device on the dish container is the third accessory device;
wherein the third accessory device with specific functions having:
at least one cone structure with a convex-concave,
a circular mark on the top cone structure,
at least one first rigid and thick square structures with a penetrative holes,
at least one second rigid and thick square structures with a penetrative holes, and
at least one penetrative hole at one side of the dish container;
Wherein the third accessory device with specific functions is used to discharge culture medium from the multifunctional Petri dish, to inject pH adjustment liquid, and extract living samples.

17. The multifunctional Petri dish recited in claims 13, 14, 15, and 16, wherein the multifunctional Petri dish with the first, second, and third accessory devices allows all biological studies and bio-industrial applications to carry out all bacteria-free biological experiments and applications under all conditions and open environments after local sterilizing.

18. The multifunctional Petri dish recited in claim 13, wherein the multifunctional Petri dishes of different volume specifications can have built-in atomization nozzles and other special devices, such as temperature sensor, pH sensor, and filter membranes or screens of various specifications.

19. The multifunctional Petri dish recited in claims 13, 14, 15, and 16, wherein the multifunctional Petri dishes allow machines to automatically add culture medium or/and replace the aerobic air in the Petri dish with filtered nitrogen or carbon dioxide gas through the accessory devices;
Whereas the multifunctional Petri dishes allow machines to automatically transfer or add living cells or strain through the accessory devices to the multifunctional Petri dish.
